(19) United States
(12) Patent Application Publication
(10) Pub. No.: US 2017/0341077 A1
(43) Pub. Date: Nov. 30, 2017

(54) MICROFLUIDIC BIOSENSOR FOR ALLERGEN DETECTION

(71) Applicant: University of Guelph, Guelph (CA)

(72) Inventors: Suresh Neethirajan, Guelph (CA); Xuan Weng, Guelph (CA)

(21) Appl. No.: 15/603,943

(22) Filed: May 24, 2017

Related U.S. Application Data
(60) Provisional application No. 62/419,696, filed on Nov. 9, 2016.
(60) Provisional application No. 62/343,287, filed on May 31, 2016.

(51) Int. Cl. B01L 3/00; G01N 30/60; G01N 33/53; G01N 33/50; G01N 30/00; G01N 35/00

(52) U.S. Cl. CPC B01L 3/502753; G01N 30/6095; G01N 33/53; G01N 30/00; G01N 2035/00099; G01N 33/50

(57) ABSTRACT

The present application relates to biosensors and methods for detecting and/or quantifying a target analyte such as a target allergen or toxin. In some embodiments, the biosensors use an allergen- or toxin-binding molecule conjugated to a fluorescent label such as a quantum dot that adheres to and is quenched by graphene oxide in the absence of the allergen or toxin.

### RELATED APPLICATIONS

This application claims priority to U.S. Provisional Patent Application No. 62/343,287 filed May 31, 2016 and U.S. Provisional Patent Application No. 62/419,696 filed on Nov. 9, 2016, the contents of which are hereby incorporated by reference in their entirety.

### INCORPORATION OF SEQUENCE LISTING

A computer readable form of the Sequence Listing “6580-P51044US02_SequenceListing.txt” (4,096 bytes), submitted via EFS-WEB and created on May 19, 2017, is herein incorporated by reference.

### FIELD

The present application relates to the detection of allergens and/or toxins, and more specifically to biosensors and methods for detecting and/or quantifying allergens and/or toxins in a liquid sample using graphene oxide and aptamer-functionalized quantum dots.

### BACKGROUND

Food allergies and poisoning have become an increasing food safety and public health concern throughout the world due to their significant effect on people's morbidity and their cost for medical visits and treatments. For allergic individuals, avoidance of the food is the only way to protect themselves against a food allergy reaction as there is no cure for food allergies (Alves et al., 2015). Although the intended presence or absence of allergens can be read from the food labels from manufacturers, undeclared allergenic substances can be inadvertently introduced into a food by uncontrolled cross-contamination, the improper use of rework, or labelling errors and cause an accidental exposure. The peanut allergy is one of the leading causes of severe food-induced allergic reactions due to its persistency and life-threatening nature. Unlike other allergies, the peanut allergy tends to be lifelong with a high risk of accidental exposure, and in highly sensitized people, trace amounts may induce an allergic reaction (Al-Muhsen et al., 2003). Among the allergenic proteins of peanuts, Ara h 1, a homotrimeric protein with a molecular weight of 65 kDa, is identified as one of the major peanut allergens presenting in a wide variety of peanuts or peanut products and shown to account for almost 95% of all peanut-allergenicity reactions (Peeters et al., 2014; Burks et al., 1991; Koppelman et al., 2004). Monitoring the presence or cross-contamination of peanut proteins is extremely important to both the food industry and sensitive individuals. With the increase in allergen awareness and regulations, many analytical methods have been developed: immunochemical methods such as ELISA (Pomés et al., 2003; Peng et al., 2013), lateral flow assay (Wang et al., 2015), DNA-based methods such as PCR (Zhang et al., 2015), and mass spectrometry (Monaci et al., 2015). Usually, these methods rely on either monoclonal or polyclonal antibodies, which are expensive to produce and possess a limited working range and shelf life (Peeters et al., 2014; Gutteridge and Thornton, 2005; Amaya-González et al., 2013; van Hengel, 2007), and the occurrence of cross-reactions is frequent. In contrast, aptamers, single-stranded oligonucleotide, or peptide sequences selected through the systematic evolution of ligands by exponential enrichment (SELEX) exhibit high affinity and specificity to various classes of target molecules. As an alternative to natural antibodies, aptamers are less expensive but more stable while still having a similar affinity to their target molecules. In addition, the synthesis and modification of aptamers are relatively easy (Zuo et al., 2013). Recently, aptamers have also been synthesized for food allergen detection (Tran et al., 2013; 2010; Nadal et al., 2013; Mairal et al., 2014; Amaya-González et al., 2014).

Biosensors have presented the potential for real-time, direct monitoring of allergens (Pilolli et al., 2013) due to their high sensitivity and selectivity while remaining relatively inexpensive, environmentally friendly, and rapid (Alves et al., 2015). However, information on the development of aptamer-based biosensors for food allergen and toxin analysis is scarcely available in the literature as it is a recent growth area. As mentioned above, aptamers have emerged as effective molecular recognition elements for ligand analysis in biosensors due to the easy modification and control without obvious deactivation. The fluorescently labeled aptamers can be used in optical sensors for molecular recognition (Cui et al., 2011).

The unique properties of nanomaterials offer excellent prospects toward the development of novel molecular diagnostic tools (He et al., 2010). Graphene oxide (GO), a single-atom-thick two-dimensional (2D) carbon nano-material, has received extensive interest in numerous biosensor applications because of its unique optical, electronic, thermal, and long-lasting biocompatibility properties (Song et al., 2013). More importantly, GO can be an efficient quencher for various fluorophores (Huang and Liu, 2012) due to the non-radioactive electronic excitation energy transfer between the fluorophore and GO (Swathi and Sebastian, 2009) and its large absorption cross section (Geim and Novoselov, 2007), providing very high quenching efficiency. In addition, GO was found to be able to interact with amino acids, peptide, and proteins by fluorescence quenching (Li et al., 2012). With its superior fluorescence quenching and adsorption capacity, GO has been increasingly used for making Förster resonance energy transfer (FRET) biosensors, aptasensors, drug-delivery vehicles, and imaging agents (Huang and Liu, 2012; Lu et al., 2015; Wang et al., 2010; Morales-Narváez and Merkoçi, 2012; Zhang et al., 2011; Lu et al., 2009). Quantum dots (Qdots) are nanomaterials that have also been increasingly used in immunoassays, fluorescent biosensing, and imaging applications (Bogomolova and Aldissi, 2011; Algar et al., 2010; Frasco and Chaniotakis, 2010) due to the chemical stability, efficient and stable fluorescence signals, and superior biological probes compared with traditional organic dyes. Aptamer conjugated Qdots for bio-recognition have been performed since 2005 (Levy et al., 2005) and later were used in the detection of protein, small biomolecules, and food-borne pathogens (Bogomolova and Aldissi, 2011). Strategies based on FRET have attracted more attention due to their inherently high sensitivity (Abachi and Noureini, 2011; He et al., 2012) and homogeneous detections (Zeng et al., 2012).

There remains a need for simple, sensitive and commercially feasible sensors for the detection of allergens and toxins.

### SUMMARY

In one aspect there is described a biosensor and associated methods for detecting and/or quantifying one or more target analytes in a liquid sample using graphene oxide (GO) and aptamer-functionalized quantum dots (QDots). In one embodiment, the target analyte is an allergen and/or toxin. The biosensor may optionally be integrated on a microfluidic platform. In one aspect, there is also described a probe composition and associated methods based on the fluorescence quenching and recovering properties of GO through the adsorption and desorption of fluorophore-conjugated antigen-binding molecules such as aptamers. As demonstrated in Example 1, the probe compositions described herein detected the major peanut allergen Ara h 1 with both a high sensitivity and selectivity. Furthermore, as demonstrated in Example 2, the probe compositions and microfluidic devices described herein detected the egg allergen lysozyme, legume allergen lupine, and the seafood toxins okadaic acid and brevetoxin with both high sensitivity and selectivity. In addition to the decreased sample/reagent consumption and rapidity introduced by using microfluidics, the device and methods described herein do not require complicated probe immobilization or tedious procedures. The fluorescence signals were measured on a miniaturized optical detector, which facilitates the portability of the device.

Accordingly, in one embodiment there is provided a probe comprising a target analyte-binding molecule such as an allergen- or toxin-binding molecule conjugated to a fluorophore. In one embodiment, the allergen- or toxin-binding molecule is an aptamer. In one embodiment, the fluorophore is a quantum dot.

In another embodiment, there is provided a probe composition comprising i) a probe comprising an target analyte-binding molecule conjugated to a fluorophore, and ii) graphene oxide. In one embodiment, the probe comprises a target allergen- or toxin-binding molecule conjugated to a fluorophore. In one embodiment, the probe adheres to graphene oxide such that the fluorophore is quenched through fluorescence energy resonance transfer (FRET). In one embodiment, the probe dissociates from graphene oxide when bound to a target analyte such as a target allergen or toxin.

In one embodiment, the allergen- or toxin-binding molecule is an aptamer, optionally a nucleic acid aptamer or a polypeptide aptamer. In one embodiment, the fluorophore is a quantum dot. In one embodiment, the allergen-binding molecule binds to a peanut allergen, optionally Ara h 1. In one embodiment, the probe comprises an antigen-binding molecule with sequence identity to SEQ ID NO: 1, wherein the antigen-binding molecule binds to Ara h 1. In one embodiment, the probe comprises an antigen-binding molecule with sequence identity to SEQ ID NO: 2, wherein the antigen-binding molecule binds to lysozyme. In one embodiment, the allergen-binding molecule binds to a legume allergen, optionally lupine. In one embodiment, the probe comprises an antigen-binding molecule with sequence identity to SEQ ID NO: 3, wherein the antigen-binding molecule binds to lupine. In one embodiment, the toxin-binding molecule binds to a seafood toxin, optionally okadaic acid or brevetoxin. In one embodiment, the probe comprises an antigen-binding molecule with sequence identity to SEQ ID NO: 4, wherein the antigen-binding molecule binds to okadaic acid. In one embodiment, the probe comprises an antigen-binding molecule with sequence identity to SEQ ID NO: 5, wherein the antigen-binding molecule binds to brevetoxin.

In another embodiment, there is provided a kit comprising a probe as described herein and graphene oxide. In one embodiment, the probe and graphene oxide are in separate containers. Optionally, the kit further comprises a microfluidic device and/or substrate for supporting a probe as described herein and graphene oxide.

In another embodiment, there is provided a biosensor comprising a probe as described herein and graphene oxide, optionally a probe composition as described herein, and a microfluidic device. In one embodiment, the biosensor comprises a plurality of probes and graphene oxide, or probe compositions, and a microfluidic device. In one embodiment, the probe compositions are contained within one or more wells in the microfluidic device. Optionally, the probe compositions are provided in separate containers.

In one embodiment, the biosensor comprises a first inlet for receiving the probe composition and a second inlet for receiving a test sample, a mixing channel in fluid communication with the first inlet and second inlet and a sensing well in fluid communication with the mixing channel. In one embodiment, the biosensor further comprises a pump for moving fluid in the mixing channel through the sensing well. In one embodiment, the biosensor further comprises an optical detector for detecting fluorescence of the fluorophore, optionally in the sensing well.

In one embodiment, the microfluidic device comprises a reaction well comprising the probe composition and a sample well for receiving a test sample. In one embodiment, the sample well is in fluid communication with a sample dispensing channel extending from the sample well to the reaction well.

In one embodiment, the probe composition is in contact with a substrate in the reaction well. The substrate may be an inert material, such as a paper substrate, optionally chromatography paper. In one embodiment, the substrate is non-fluorogenic such as to not interfere with the detection of the fluorophore.

In one embodiment, the microfluidic device comprises a plurality of sample dispensing channels extending radially from the sample well to a plurality of reaction wells. Different reaction wells on the microfluidic device may comprise the same or different probes for the detection of the same or different target analytes. In one embodiment, a first reaction well comprises a first probe comprising a first allergen- or toxin-binding molecule and a second reaction well comprises a second probe comprising a second allergen- or toxin-binding molecule, wherein the first probe and second probe bind to different allergens or toxins. In one embodiment, the microfluidic device comprises at least two reaction wells comprising probes that bind to the same allergen or toxin.

In one embodiment, the microfluidic device comprises one or more waste wells in fluid communication with the one or more reaction wells.

In one embodiment, all or part of the microfluidic device is made of glass and/or an elastomeric material such as polydimethylsiloxane (PDMS).

In one embodiment, the biosensor further comprises an optical detector. In one embodiment, the optical detector comprises an excitation light source and a photodiode for measuring fluorescence of the fluorophore.

In another embodiment, there is provided a method for detecting and/or quantifying a concentration of a target allergen or toxin in a sample. In one embodiment, the method comprises contacting a probe composition as described herein with the sample and detecting a level of fluorescence of the probe composition in contact with the sample. In one embodiment, the level of fluorescence is proportional to the concentration of target allergen or toxin in the sample. In one embodiment, the method comprises detecting a level of fluorescence of the probe composition prior to contacting the probe composition with the sample. Optionally, the method further comprises comparing the level of fluorescence of the probe composition in contact with the sample to one or more control levels. In one embodiment, each control level is representative of a pre-determined concentration of the target allergen or toxin in a control sample.

In one embodiment, the biosensors and methods described herein are for the detection of allergens and toxins using a portable device. In another embodiment, the biosensors and methods described herein are for the detection of allergens and toxins in a laboratory setting.

Other features and advantages of the present application will become apparent from the following detailed description. It should be understood, however, that the detailed description and the specific examples while indicating embodiments of the application are given by way of illustration only and the scope of the claims should not be limited by the embodiments set forth in the examples, but should be given the broadest interpretation consistent with the description as a whole.

### BRIEF DESCRIPTION OF THE DRAWINGS

The present application will now be described in greater detail with reference to the drawings in which:

FIG. 1 shows (A) Schematic of the sensing mechanism of the Qdots-aptamer-GO quenching system. (B) Schematic diagram of microfluidic chip design (not to scale). The microfluidic chip had two inlets for loading the Qdots-aptamer-GO probe mixture and the Ara h 1 sample, respectively. The main channel of 200 μm wide and 60 μm deep consisted of a mixing/incubation channel, a sensing well, and a capillary pump at the end. The long and zigzag-shaped channel was designed to enhance the mixing effect. The “diamond”-shaped well was the sensing well aligned to the sensing window of the Si photodiode. The flow was driven by the capillary forces.

FIG. 2 shows characterization of Qdots-aptamer probes. (A) TEM images of Qdots-streptavidin conjugates; (B) TEM images of Qdots-aptamer probes. (C) Particle size distribution of Qdots-streptavidin conjugates and Qdots-aptamer probes by DLS. The mean hydration diameter of the QDs and QDs-aptamer are 21.9 nm and 47.9 nm, respectively. (D) Fluorescence spectra of Qdots and Qdots-aptamer probes.

FIG. 3 shows optimization of (A) Incubation time of Qdots conjugation with Ara h 1 aptamer; (B) Aptamer concentrations in effecting quenching and recovery performance; (C) GO concentrations in effecting quenching and recovery performance; (D) Quenching and recovery time.

FIG. 4 shows fluorescence spectra (A) and fluorescence images (B) of Ara h 1 standard solution of various concentrations by Qdots-aptamer-GO system before quenching (BQ), after quenching (AQ), and recovery.

FIG. 5 shows (A) standard calibration curve for the relative voltage output change depending on fluorescence intensity caused by Ara h 1 concentration ranging from 200˜2000 ng/mL. Each data point was obtained from three independent measurements. (B) Fluorescence spectra and (C) relative voltage output difference depending on fluorescence intensity in the presence of Ara h 1, Ara h2, and Ara h3 of 1000 ng/mL, respectively.

FIG. 6 shows a schematic of one embodiment of a biosensor described herein that includes a PDMS/paper microfluidic device for food allergens and toxins detection (not to scale). The device includes a sample loading well and four reaction wells with associated dispensing channels. Four pieces of circle chromatography paper modified with GO-aptamer-QDs by physical deposition were housed at the bottom of the reaction wells, respectively. The reaction wells were open to the air by a channel at the bottom. The diameters of the reaction well, reaction wells and the waste wells were 4 mm, 3 mm and 2 mm, respectively. The sample dispensing channel is 200 μm in width and 80 μm in depth. The overall size of the microfluidic chip is 25 mm×75 mm.

FIG. 7 shows fluorescence spectra upon the reaction of aptamer functionalized QDs with a series of standard solutions of (A) egg white lysozyme; (B) lupine; (C) okadaic acid; and (D) brevetoxin at the stages of before quenching (BQ), after quenching (AQ) and recovery (RC).

FIG. 8 shows on-chip tests of the fluorescence images of lysozyme standard solutions of various concentrations at after quenching (AQ) and recovery (RC) stages.

FIG. 9 shows standard calibration curves for the fluorescence intensity changes depending on the concentrations of the targets. (A) egg white lysozyme; (B) lupine; (C) okadaic acid; and (D) brevetoxin.

### DETAILED DESCRIPTION

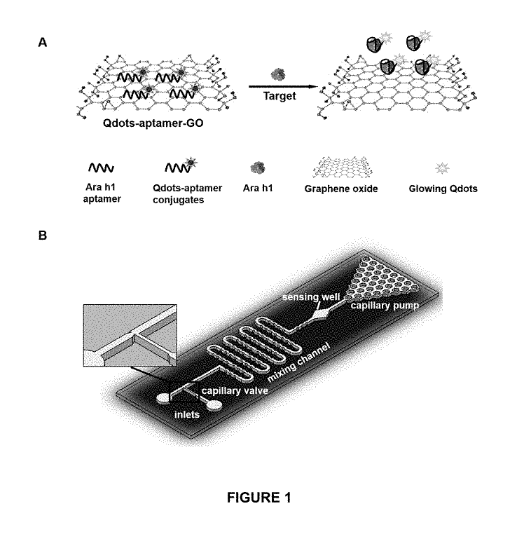

The inventors have developed a biosensor and associated methods useful for detecting a target analyte in a fluid sample. In a preferred embodiment, the target analyte is an allergen or toxin, optionally a food allergen or toxin. In one embodiment, a probe is used that comprises an allergen- or toxin-binding molecule conjugated to a fluorophore in a composition comprising graphene oxide. Without being limited by theory and as shown in FIG. 1A, it is believed that the probe adheres to graphene oxide in the absence of a target allergen or toxin such that the fluorophore is quenched through fluorescence energy resonance transfer (FRET). The presence of a target allergen or toxin causes the antigen-binding molecule to bind to the target allergen or toxin such that probe dissociates from graphene oxide, reducing the quenching effect and resulting in detectable fluorescence.

In one embodiment, there is provided a probe comprising an allergen- or toxin-binding molecule conjugated to a fluorophore. In one embodiment, the allergen- or toxin-binding molecule is an antibody. In one embodiment, the allergen- or toxin-binding molecule is an aptamer and the fluorophore is a quantum dot.

In one embodiment, there is provided a probe composition comprising a probe comprising an allergen- or toxin-binding molecule conjugated to a fluorophore, and graphene oxide. In one embodiment, the probe adheres to graphene oxide in the composition such that the fluorophore is quenched through fluorescence energy resonance transfer (FRET). In one embodiment, the association constant between the probe and graphene oxide is lower than the association constant between the probe and the target allergen or toxin. In one embodiment, the probe dissociates from graphene oxide and binds to the target allergen or toxins in the presence of the target allergen or toxin.

In one embodiment, the fluorophore is quenched by FRET when interacting with graphene oxide. In one embodiment, the fluorophore is a quantum dot. In one embodiment, the fluorophore absorbs light at higher energy wavelengths and emits light at lower energy wavelengths.

In one embodiment, the allergen- or toxin-binding molecule is an aptamer or antibody. Preferably, the allergen- or toxin-binding molecule selectively binds a particular allergen or toxin, such as an allergen or toxin associated with the presence of a particular food or which may cause an allergic or toxic reaction in a subject.

As used herein, the term “allergen” refers to a substance that causes an abnormally vigorous immune response within a subject. In one embodiment, an allergen is an antigen capable of stimulating a type-I hypersensitivity reaction in atopic individuals through Immunoglobulin E (IgE) responses. In some embodiments, an allergen may also be a toxin. Optionally, the allergen may be an allergen that is present in a food or edible substance. Examples of allergens include, but are not limited to peanut allergens, egg allergens, legume allergens, milk allergens, seafood allergens, mustard allergens, sesame allergens, soy allergens, tree nut allergens and wheat allergens.

As used herein, the term “toxin” refers to a substance produced by a living cell or organism that is capable of causing disease on contact with a subject. In some embodiments, a toxin may cause disease in a subject by interacting with biological macromolecules such as enzymes or cellular receptors within the subject. In some embodiments a toxin may also be an allergen. Examples of toxins include, but are not limited to, mycotoxins such as flatoxins, ochratoxin A, ergot alkaloids, fumonisins, patulin, trichothecenes (such as deoxynivalenol which is also known as vomitoxin) and zearalenone, toxins present in seafood such as okadaic acid and brevetoxin, and anatoxins such as toxins produced by cyanobacterial algae blooms.

For example, in one embodiment, the allergen-binding molecule selectively binds to a peanut allergen. In one embodiment, the peanut allergen is Ara h 1. In one embodiment, the allergen binding molecule is an aptamer having sequence identity to SEQ ID NO: 1. In one embodiment, the allergen binding molecule comprises or consists of a nucleic acid sequence with at least 70%, 75%, 80%, 85%, 90%, 95% or 100% identity to SEQ ID NO: 1.

In another embodiment, the allergen-binding molecule selectively binds to an egg allergen. In one embodiment, the egg allergen is lysozyme. In one embodiment, the allergen-binding molecule is an aptamer having sequence identity to SEQ ID NO: 2. In one embodiment, the allergen-binding molecule comprises or consists of a nucleic acid sequence with at least 70%, 75%, 80%, 85%, 90%, 95% or 100% identity to SEQ ID NO: 2.

In another embodiment, the allergen-binding molecule selectively binds to a legume allergen. In one embodiment, the legume allergen is lupine. In one embodiment, the allergen-binding molecule is an aptamer having sequence identity to SEQ ID NO: 3. In one embodiment, the allergen-binding molecule comprises or consists of a nucleic acid sequence with at least 70%, 75%, 80%, 85%, 90%, 95% or 100% identity to SEQ ID NO: 3.

In another embodiment, the probe comprises an allergen-binding molecule that selectively binds to an allergen selected from peanut allergens, egg allergens, legume allergens, milk allergens, seafood allergens, mustard allergens, sesame allergens, soy allergens, tree nut allergens and wheat allergens. In one embodiment, the legume allergens are lupin allergens.

In another embodiment, the probe comprises a toxin-binding molecule. In one embodiment, the toxin-binding molecule selectively binds to a seafood toxin. In one embodiment, the seafood toxin is okadaic acid or brevetoxin. In one embodiment, the toxin-binding molecule is an aptamer having sequence identity to SEQ ID NO: 4 or 5. In one embodiment, the allergen-binding molecule comprises or consists of a nucleic acid sequence with at least 70%, 75%, 80%, 85%, 90%, 95% or 100% identity to SEQ ID NO: 4 or 5.

In one embodiment, the probe comprises a toxin-binding molecule that selectively binds to a toxin selected from mycotoxins, toxins present in seafood such as okadaic acid and brevetoxin, and anatoxins.

Antibodies and/or aptamers that selectively bind to a target analyte such as an antigen or toxin may readily be prepared by a skilled person using methods known in the art. For example, methods for generating nucleic acid aptamers that bind to a target analyte include methods that use SELEX (Systematic Evolution of Ligands by EXponential enrichment) or other methods that include generating a library of nucleic acid sequences and then sequentially selecting for sequences that bind to a target analyte, such as by affinity chromatography.

Different methods known in the art may be used to conjugate the antigen-binding molecule and fluorophore. For example, in one embodiment, the antigen-binding molecule and fluorophore are covalently bound. In one embodiment, the antigen-binding molecule and fluorophore are bound using biotin and streptavidin.

The probe and/or probe compositions described herein may be used in association with a microfluidic device to facilitate the detection of one or more target allergens and/or toxins. For example, in one embodiment there is provided a biosensor comprising a probe, graphene oxide and/or probe composition as described herein and a microfluidic device. Optionally, there is provided a kit comprising i) a probe, graphene oxide and/or probe composition in one or more separate containers and ii) the microfluidic device. In some embodiments, the biosensor is for the detection of a plurality of different target allergens and/or toxins and comprises a plurality of different probes, graphene oxide and/or probe compositions as described herein.

An exemplary microfluidic device useful for detecting allergens and/or toxins using a probe as described herein is shown in FIG. 1B. In one embodiment, the microfluidic device comprises a microchannel for entry and exit of a liquid sample and a probe, graphene oxide and/or probe composition.

A variety of different kinds of samples containing or suspected of containing a target analyte such as an allergen of toxin may be detected and/or quantified using the microfluidic device and methods described herein. In one embodiment, the sample is an environmental sample such as a water sample. In one embodiment, the sample is a sample from a manufacturing or processing facility. In one embodiment, the sample is a food sample. In one embodiment, the sample is a diagnostic sample obtained from a subject such as a human or other animal. In one embodiment, the diagnostic sample comprises a bodily fluid, such as urine, saliva, blood, mucus, faeces or spinal fluid.

In one embodiment, the microfluidic device comprises a first inlet for receiving a probe, graphene oxide and/or a probe composition and a second inlet for receiving a test sample. In one embodiment, the microfluidic device comprises a mixing channel in fluid communication with the first inlet and second inlet. In one embodiment, the microfluidic device comprises a sensing well, optionally a portion of the mixing channel. Preferably, the sensing well is made of optically transparent material to allow for the detection of fluorophores within the sensing well.

In one embodiment, the microfluidic device further comprises a pump for moving fluid from the first inlet and/or second inlet through the mixing channel to the sensing well. Optionally, the pump is a capillary pump or another pump suitable for use in microfluidic devices.

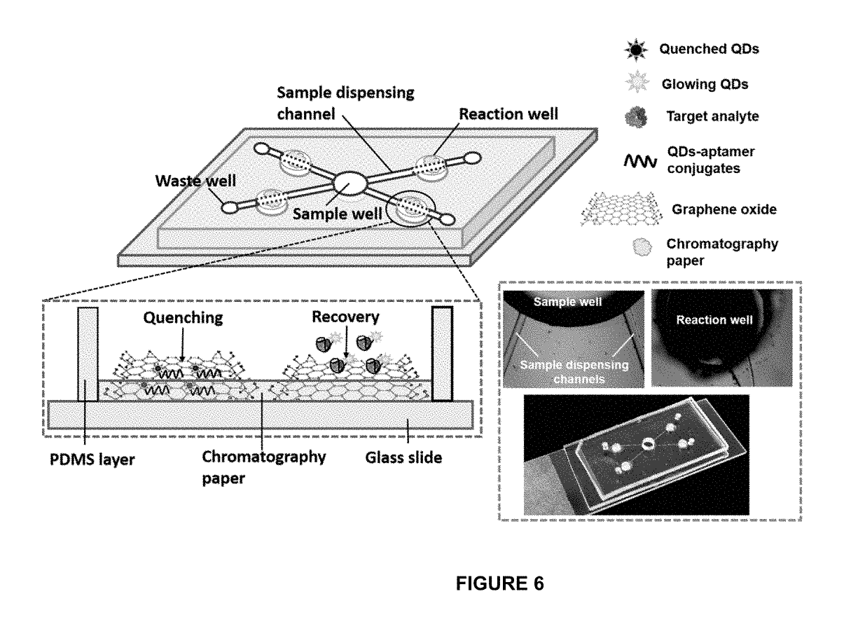

Another exemplary microfluidic device useful for detecting allergens and/or toxins using one or more probes as described herein is shown in FIG. 6. In one embodiment, the microfluidic device comprises a sample well for receiving a test sample. One or more reaction wells are in fluid communication with the sample well via one or more sample dispensing channels. In operation, a fluid sample introduced into the sample well is drawn into the one or more reaction wells through the sample dispensing channels through capillary action.

In one embodiment, the one or more reaction wells contain a probe composition. Optionally, the probe composition is associated with a substrate and/or in contact with a substrate in the reaction well. In one embodiment the substrate is an inert substrate that is not fluorogenic and/or does not interfere with the quenching of the fluorophore by graphene oxide or the detection of the fluorophore in the presence of a target analyte. For example, in one embodiment the substrate is a paper substrate, optionally chromatography paper. In one embodiment, the chromatography paper is coated with a predetermined amount of a probe composition prior to positioning the chromatography paper within the reaction well. Preferably, the one or more reaction wells are made of optically transparent material to allow for the detection of fluorophores within the reaction well.

The microfluidic devices described herein may be made out of different elastomeric materials known in the art such as plastic, silicone and/or glass. In one embodiment, the microfluidic device is made of polydimethylsiloxane (PDMS). In one embodiment, the microfluidic device is made using lithographic techniques for microfabrication known in the art, such as photolithography and/or soft lithography.

In one embodiment, the microfluidic device comprises two or more layers. In one embodiment, the microfluidic device comprises two or more layers, wherein the top layer comprises a sample inlet or sample well, one or more sample outlets and/or associated sample dispensing channels. In one embodiment, the bottom layer comprises one or more reaction wells.

In one embodiment, the biosensor comprises an optical detector. In one embodiment, the optical detector comprises an excitation light source and a photodiode for measuring fluorescence of the fluorophore. A suitable optical detector may be selected for a biosensor as described herein based on the fluorescent properties of the fluorophore conjugated to the analyte-binding molecule.

The biosensors and methods described herein may be integrated into different devices or protocols in order to detect and/or quantify a target analyte such as a target allergen or toxin in a sample. In one embodiment, the biosensor is in a hand-held system for detecting a target allergen or toxin in a fluid sample. In one embodiment, the biosensor is an in-line system for detecting a target allergen or toxin in a fluid sample.

Also provided is a method for detecting and/or quantifying a concentration of a target analyte in a sample, optionally a target allergen or toxin. In one embodiment, the method comprises contacting a probe, graphene oxide and/or a probe composition as described herein with a sample and detecting a level of fluorescence of the probe composition in contact with the sample. In one embodiment, the level of fluorescence is proportional to the concentration of target allergen or toxin in the sample. In one embodiment, the method comprises comparing the level of fluorescence of the probe composition in contact with the sample to one or more controls.

In one embodiment, the methods described herein include detecting a level of fluorescence of the probe composition prior to contacting the probe composition with the sample. In one embodiment, an increase in the level of fluorescence after contacting the probe composition with the sample indicates the presence of the target allergen or toxin in the sample.

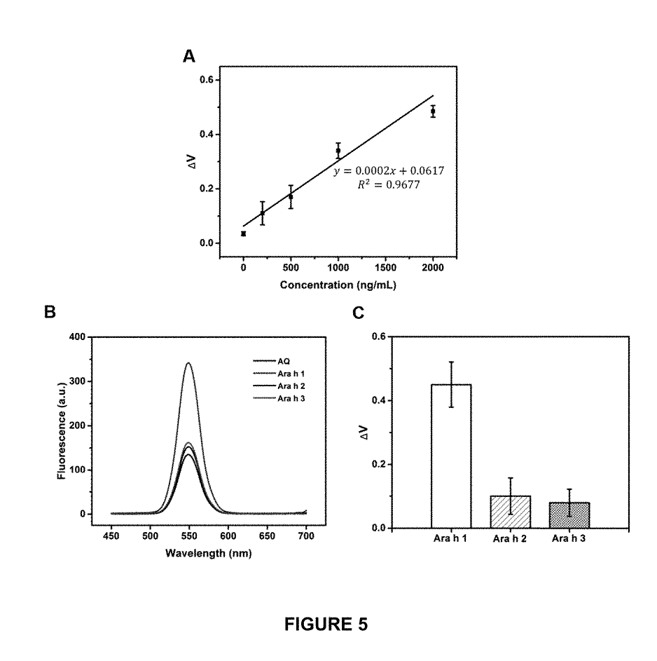

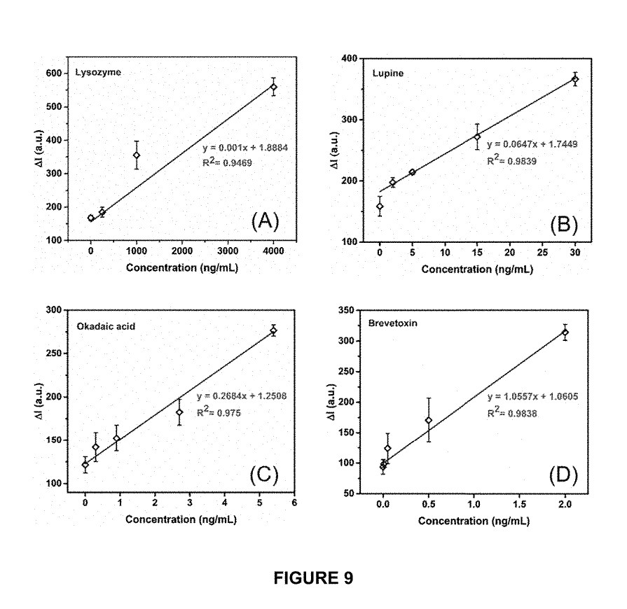

In one embodiment, the method comprises comparing the level of fluorescence of the probe composition in contact with the sample to one or more control levels. In one embodiment, the each control level is representative of a pre-determined concentration of the target allergen or toxin in a control sample. As shown in FIG. 5A and FIG. 9, a standard calibration curve may be generated in order to estimate the concentration of a target allergen or toxin in a sample as described herein. Optionally, the methods described herein include detecting a level of fluorescence of the probe composition prior to contacting the probe composition with the sample and comparing the change in the level of fluorescence to one or more control levels.

In one embodiment, the method comprises using a biosensor comprising a microfluidic device as described herein to detect one or more target allergens and/or toxins. For example, in one embodiment, the method comprises introducing a probe, graphene oxide and/or probe composition as described herein into a first inlet, introducing a sample into a second inlet, allowing the sample to come into contact with the probe, and detecting a level of fluorescence. In another embodiment, the method comprises introducing a sample into a sample well, allowing the sample to contact a probe composition as described herein in a reaction well, and detecting a level of fluorescence.

In another embodiment, there is also provided a mixture of two or more probes that each selectively binds to a different target antigen and are conjugated to a different fluorophores. Accordingly, the biosensors and methods described herein may be used for the simultaneous detection of two or more target allergens and/or toxins in a sample or for the detection of a single target allergen or toxin in a sample. Alternatively or in addition, the biosensors and methods described herein may include a plurality of inlets, wells, mixing channels and/or dispensing channels for the detection of different target analytes using different probes.

The methods, devices and compositions described herein may be used for detecting and/or quantifying a variety of different target analytes in a sample. For example, in one embodiment the methods, devices and compositions described herein are useful for detecting and/or quantifying one or more target allergens or toxins. In one embodiment, the target allergen is selected from peanut allergens, egg allergens, legume allergens, milk allergens, seafood allergens, sesame allergens, soy allergens, tree nut allergens and wheat allergens. In one embodiment, the target toxin is selected from mycotoxins, okadaic acid and brevetoxin.

The following non-limiting examples are illustrative of the present application:

### EXAMPLES

### Example 1

### A Microfluidic Biosensor Using Graphene Oxide and Aptamer-Functionalized Qantum Dots for Peanut Allergen Detection

The increasing prevalence of food allergies and the intake of packaged foods in the past two decades have prompted the need for more rapid, accurate, and sensitive assays to detect potential allergens in food in order to control the allergen content. Most of the commercial analytical tools for allergen detection rely on immunoassays such as ELISA. However, ELISA can be time-consuming and expensive. Biosensors appear as a suitable alternative for the detection of allergens because they are rapid, highly sensitive, selective, less expensive, environmentally friendly, and easy to handle. Here, there is described a microfluidic system integrated with a quantum dots (Qdots) aptamer functionalized graphene oxide (GO) nano-biosensor for simple, rapid, and sensitive food allergen detection. The biosensor utilized Qdots-aptamer-GO complexes as probes to undergo conformational change upon interaction with the food allergens, resulting in fluorescence changes due to the fluorescence quenching and recovering properties of GO by adsorption and desorption of aptamer-conjugated Qdots. This one-step ‘turn on’ homogenous assay in a ready-to-use microfluidic chip took ˜10 min to achieve a quantitative detection of Ara h 1, one of the major allergens appearing in peanuts. The integration of a microfluidics platform in a miniaturized optical analyzer provides a promising way for the rapid, cost-effective, and accurate on-site determination of food allergens. This biosensor may also be extended to the detection of other food allergens and toxins with a selection of corresponding aptamers. For example, additional allergen- or toxin-binding aptamers are described in Tran et al., 2013; 2010; Nadal et al., 2013; Mairal et al., 2014; Amaya-González et al., 2014 and Cruz-Aguado and Penner, 2008, hereby incorporated by reference in their entirety.

### Materials and Methods

The Ara h 1 aptamer synthesis was obtained from IDT technologies (Coralville, Iowa, USA) and had the following 80 base pairs sequence:

(SEQ ID NO: 1)
5'TCGCACATTCCGCTTCTACCGGGGGGGTCGAGCGAGTGAGCGAATCT
GTGGGTGGGCCGTAAGTCCGTGTGTG CGAA 3'

The 5′end was modified with biotin. Ara h 1, Ara h2, and Ara h3 standards and the Ara h 1 ELISA kit were purchased from INDOOR Biotechnologies Inc. (Charlottesville, Va., USA). Commercially available CdSe Qdots modified with covalently attached streptavidin (Qdot® 545 ITK™ Streptavidin Conjugates) were purchased from Invitrogen Life Technologies (Burlington, ON, Canada). Polydimethylsiloxane (PDMS, Sylgard, 184) was obtained from Dow Corning (Midland, Mich., USA). Graphene oxide, phosphate-buffered saline (PBS), and all other mentioned chemicals and solvents were purchased from Sigma-Aldrich (Oakville, ON, Canada). Unless otherwise noted, all solutions were prepared with ultrapure DI water.

### Preparation of Aptamer-Conjugated Quantum Dots

The dried aptamer pellet was firstly resuspended in TE buffer (10 mM Tris HCl, 0.1 mM EDTA, pH 8.0) to achieve a 100 μM concentration. The resuspended aptamer was incubated at room temperature for 30 minutes, created aliquots, and stored at −20° C. Prior to use, the aptamer was diluted to the working concentration in the folding buffer (1 mM MgCl₂, 1×PBS, pH 7.4) and heated at 85° C. for 5 min. The cooling-down dilutions were ready for use.

Streptavidin-conjugated quantum dots of 2 μM were mixed with 3.2 μM 5′-biotin aptamers in a 200 μL PBS (pH 7.4, 0.01 M). The quantum dots were covalently linked to 5′-biotin aptamers via streptavidin-biotin interaction. The mixture was incubated under gentle mixing at room temperature. After incubation, the Qdots-aptamer conjugates mixture was subjected to ultrafiltration with PBS (three times, 15 min at 6000 rpm, cut-off filter 50 kD) to remove the excess of the unbound aptamer. The conjugates were finally resuspended in a 0.01 M PBS solution (pH 7.4) for Ara h 1 detection.

### Preparation of Qdots-Aptamer-GO Quenching System

GO was diluted in ultrapure DI water and mixed with the Qdots-aptamer conjugates solution. The excellent dispersibility is very critical for its sufficient interaction with aptamer molecules. The BSA solution was added into the mixture at a final concentration of 0.5%. The mixture of Qdots-aptamer conjugates and GO was incubated for a period of time to quench the fluorescence of the Qdots-aptamer conjugates. Optimal GO concentration and quenching time were investigated.

### Detection of Ara h 1

Different concentrations of Ara h 1 standard solution or food samples were added into the Qdots-aptamer-GO quenching system with gentle shaking. The mixtures were then incubated at room temperature. Incubation times of 5, 10, 15, and 20 min were investigated. Fluorescence spectra of the mixtures were measured to evaluate the Qdots fluorescence recovery and make the standard curve. The schematic of the sensing mechanism is shown in FIG. 1A. Fluorescence of Qdots is quenched via FRET process between the Qdots-aptamer probes and GO due to their self-assembly through specific π-π interaction. In the presence of the target Ara h1 protein, the association constant between the Qdots-aptamer probes and Ara h 1 is bigger than that between the Qdots-aptamer probes and GO, resulting in the release of the Qdots-aptamer probes from GO and thus the recovery of the fluorescence of Qdots.

### Characterization of the Qdots-Aptamer Probes

The modification of the fluorescent Qdots and Ara h 1 aptamers were bridged with a streptavidin-biotin site-specific bioconjugation system via the extraordinarily high affinity of streptavidin homo-tetramers for biotin. Then, the Qdots-aptamer probes were characterized by fluorescence spectra (Synergy H4 Hybrid Multi-Mode Microplate Reader, Biotek, Winooski, Vt., USA), TEM (Tecnai G2 F20, FEI, Hillsboro, Oreg., USA), and a DLS system (ZetaPlus Zeta potential analyzer, Brookhaven Instruments Corporation, Holtsville, N.Y., USA).

### Optimization

Optimization was firstly performed by investigating the molar ratio (1:1, 1:4, and 1:8) of between Qdots and Ara h 1 aptamer and incubation time (30 min, 4 hr, and 12 hr) during the Qdots modification. In the sensing processing, concentrations of graphene oxide and the incubation times for quenching and recovery affect not only the quenching rate but also the recovery efficiency, which can directly affect the detection sensitivity. Hence, the optimization of GO concentration (0˜0.1 mg/mL) and quenching/recovery time (0˜15 min) were also investigated.

Microplate reader and fluorescence microscopic detection

To validate the performance of the Qdot-aptamer-GO quenching system, fluorescence analysis was conducted by fluorescent spectra analysis on a microplate reader (Synergy H4 Hybrid Multi-Mode Microplate Reader, Biotek, Winooski, Vt., USA) and fluorescent imaging on a fluorescent microscopy (Nikon Eclipse Ti, Nikon Canada Inc., Mississauga, ON, Canada).

Microfluidic biochip fabrication and signal capture

The schematic design of the microfluidic biochip is shown in FIG. 1B. The microfluidic chip consisted of two inlets, a mixing/incubation channel, a sensing well, and a capillary pump. The powerless sampling can be generated by this “hexagons” capillary pump by splitting the capillary pump into hundreds of smaller parallel microchannels; hence, the liquid could be sucked into the microfluidic channel by the capillary force. The design at the entrance of the mixing/incubation channel was a capillary-driven retarding valve (Mohammed and Desmulliez, 2013), which helped to avoid air capture in the microchannel when dispensing the Qdots-aptamer-GO probe mixture and the Ara h 1 sample into the inlets. In addition, to enhance the mixing effect of the mixture in the microchannel, a zigzag microchannel was designed. The total length of the mixing microchannel was around 10 cm.

The microfluidic chips were fabricated by using the standard soft lithography technique. A master-carrying microchannel mold was firstly prepared by spin-coating a thin film of SU-8 negative photoresist (MicroChem, Westborough, Mass., USA) on a silicon wafer followed by pre-baking. The photoresist film covered with a mask bearing the desired microchannel geometry was then exposed to UV light. After post-baking and developing, a master mold could be obtained. The second step was making the PDMS chips. A degassed mixture of PDMS prepolymer and curing agent (10:1, w/w) was poured over the master mold and cured at 75° C. for about 4 hr. After the incubation, the PDMS replica was peeled off the master mold, and the inlets were punched. The PDMS microchannel was placed on a glass slide filled with 10 mg/ml BSA solution and left to dry at room temperature, which was performed to prevent the non-specific adsorption of the desired proteins onto the channel wall. Then, the PDMS microchannel was peeled away from the slide and sealed against a clean glass slide after the oxygen plasma was treated for 40 s.

To make the whole sensing system capable of on-site detection, the fluorescence signals were measured by a miniaturized optical detector. The details of the optical detector can be found in Weng et al., 2015, hereby incorporated by reference in its entirety. Briefly, the miniaturized optical detector consisted of LED (447.5 nm, Luxeon Rebel, Luxeon Star LEDs, Brantford, ON, Canada) mounted on the top to provide excitation light and a low-noise, high-sensitivity Si photodiode (Hamamatsu, Bridgewater, N.J., USA) mounted on the bottom for emission light capture. The PDMS well was placed in between and aligned to both the LED and the sensing window of the photodiode. Excitation (445/20-25 nm, Semrock, Rochester, N.Y., USA) and emission (531/20-25 nm, Semrock, Rochester, N.Y., USA) optical filters were used to reduce interference. All components were assembled in a container (10×6×5 cm³) to block ambient light. The collected light intensity signal by the Si photodiode was then digitized and transferred to a PC for storage and analysis by a programmable microcontroller (Arduino Uno, SparkFun Electronics, Niwot, Colo., USA).

Based on the optimization, 10 μL of Qdots-aptamer probes with a GO solution with a final concentration at 0.05 mg/mL and 10 μL of Ara h 1 standard solution and the food sample solution were added in the inlets of the microfluidic device, respectively. The fluorescence intensity was measured immediately when the mixture flew into the sensing well as a reference by the photodiode. After 5 minutes of recovery, the fluorescence intensity was measured and recorded again, and the difference was used to differentiate the Ara h 1 concentration.

### Results and Discussion

### Characterization of the Qdots-Aptamer Probes

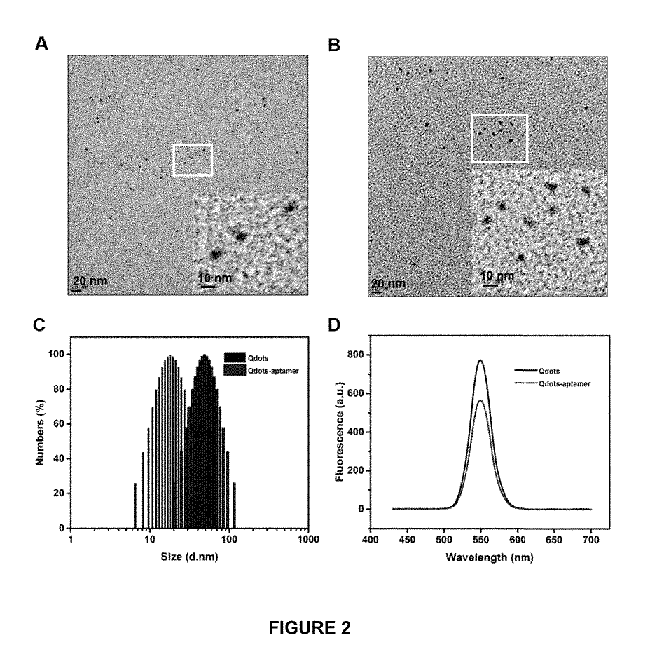

Conjugation may cause a change in the nanoparticle size, zeta potential, and so on. Hence, the Qdots-aptamer probes were characterized first. The morphology of the Qdots before and after conjugation with the aptamer were characterized by TEM imaging as shown in FIG. 2A and FIG. 2B. Both nanoparticles showed a cone-like shape and were well dispersed, but no significant changes were observed in the particle size (˜10 nm) of Qdots-aptamer probes. The hydration diameters of the Qdots before and after conjugation were measured and compared by dynamic light scattering (DLS) analysis. As shown in FIG. 2C, the mean hydration diameter of the nanoparticle increased from 21.9 nm and 47.9 nm while the zeta (ζ) potential values increased from −34.3±3.0 mV to −42.2±4.1 mV. Since the modifier streptavidin and Ara h 1 aptamer could not be observed by TEM, the particle sizes detected by TEM were smaller than those detected by DLS. In addition, a decrease of the fluorescence intensity of the Qdots before and after conjugation was also observed (FIG. 2D). The concentration of Qdots in both solutions was 0.2 μM. These results indicated that Qdots were successfully bound with Ara h 1 aptamers.

### Optimization and Sensing Mechanism Validation

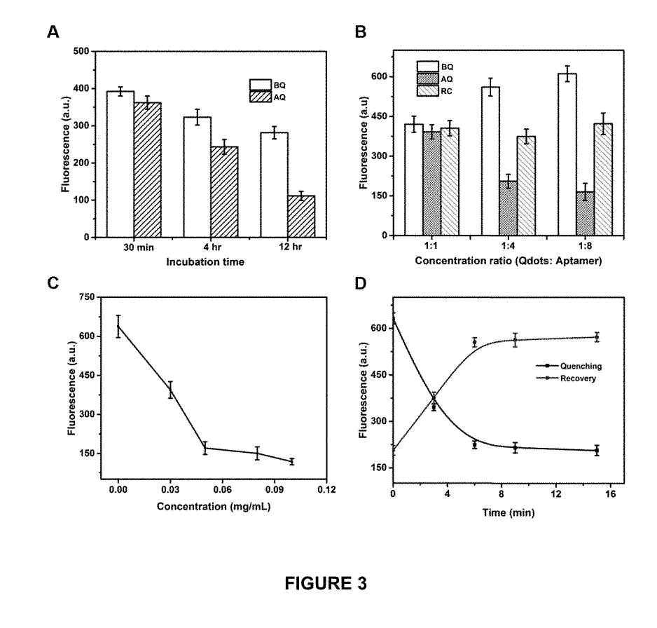

The preparation of the Qdots-aptamer probes was the key for the sensing event, which significantly affected the fluorescence quenching and recovery; hence, the conjugation of Ara h 1 aptamer onto the Qdots was studied. The incubation time and aptamer concentration were optimized. 30 minutes, 4 h, and 12 h of incubation were tested, and the fluorescence response before quenching (BQ) and after quenching (AQ) were investigated as shown in FIG. 3A. The overnight incubated probes presented a significant quenching effect compared to the other two. Three different ratios of concentration, 1:1, 1:4, and 1:8, between Qdots and Ara h 1 aptamers were tested by observing the fluorescence intensities before quenching (BQ), after quenching (AQ), and after recovery (RC). The result of detection of Ara h 1 of 2000 ng/mL was shown in FIG. 3B. After the addition of graphene oxide of the same concentration, fluorescence quenching occurred for all the three concentrations. However, low-quenched fluorescence and high-recovered fluorescence would provide high sensitivity. From the result, we see that the higher ratio of 1:8 presented lower and higher fluorescence in quenching and recovery. In addition, we found that when the concentration ratio was bigger than 1:8, no significant improvement was found. Therefore, the ratio of 1:8 and overnight incubation were used in the following tests. To obtain the optimized quenching effect, the concentrations of GO were also investigated. As shown in FIG. 3C, bigger fluorescence quenching occurred with the increasing of the concentration of GO ranging from 0.03mg/mL to 0.1 mg/mL. From the result, 0.05 mg/mL presented the superior quenching effect, while a concentration higher than 0.05 mg/mL did not show a significant difference. However, a distinctive lower fluorescence intensity was observed at the concentration of 0.1 mg/mL, which may be caused by the turbidity due to the high concentration of GO. Hence, a final concentration of 0.05 mg/mL of GO in the mixture was used. The assay time depended on the quenching and recovery time; hence, the incubation times for fluorescence quenching and recovery were investigated, the result of which was shown in FIG. 3D. Fluorescence intensities in the processes of quenching and recovery were measured at the time points at 0, 3, 6, 9, and 15 min, respectively. We found that the significant changes of quenching and recovery occurred within the first 6 min of the both processes; hence, 5 min was used as the quenching and recovery time. Therefore, 10 min was considered the total assay time for a single test.

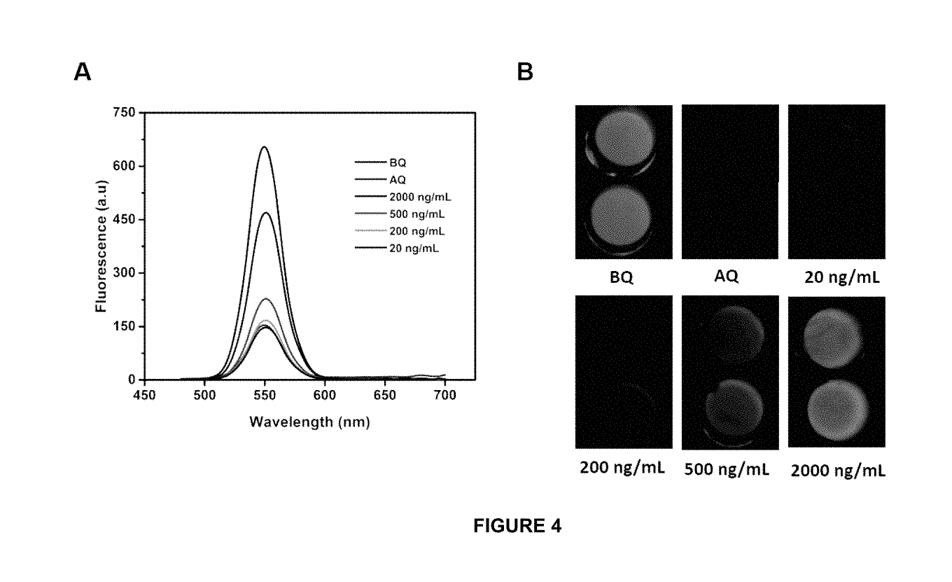

With the optimized settings obtained above, full assays on various concentrations of Ara h 1 standard solution ranging from 20 ng/mL to 2000 ng/mL were conducted in a 384 microplate well and read by a microplate reader to validate the sensing mechanism. The fluorescence spectra and fluorescence images were shown in the FIG. 4A and FIG. 4B.

### Detection by Optical Detector

The time-dependent fluorescence changes upon the reaction of Qdots-aptamer-GO with Ara h 1 standard solution were investigated by a custom-designed miniaturized optical detector, which is shown in FIG. 5A. Based on the optimization results, 5 min was taken as the quenching time and fluorescence recovery time. To minimize the error, the output of the photodiode after quenching and recovery were recorded, and the relative difference between these two values was used to differentiate the sample concentration. As shown in FIG. 5A, the output of the photodiode increased as the concentration of Ara h 1 increased, which indicates the recovered fluorescence intensity of the Qdots was intensified. The relative differences obtained by detecting Ara h 1 standard solution of various concentrations were used to make the standard curve. An R² value of 0.9677 was found for the linear response region between 200 ng/mL and 2000 ng/mL with detection limits of 56 ng/mL (Thomsen et al., 2003).

### Specificity and Food Sample Detection

Ara h 1, Ara h2, and Ara h3 are all the major allergens presented in peanuts. Specificity analysis was performed by an investigation of the system response to the possible interferences introduced by Ara h2 and Ara h3. Ara h 1, Ara h2, and Ara h3 of the same concentration at 1000 ng/mL were assayed by the Qdots-aptamer-GO system. As shown in FIG. 5B and FIG. 5C, little fluorescence recovery was found by Ara h2 and Ara h 3 compared to the significant fluorescence intensity increase by Ara h 1, which demonstrated the high selectivity and specificity of our system in determining Ara h 1.

To validate the performance of our optical biosensor, when working on the real food sample was assayed, Ara h 1 in a biscuit was detected by our method and a commercial ELISA kit for a comparison. Food sample preparation was conducted by following the procedure indicated in the manual of the commercial kit. Briefly, 1 g of the homogenized biscuit sample was mixed with 20 mL of the sample extraction solution provided by the kit and followed by incubation in a water bath at 60° C. for 15 min. After being cooled down under room temperature, the solution mixture was centrifuged at 2,000×g for 10 min. The supernatant of the mixture was filtered through a filter syringe (GHP Membrane Disc Filters, VWR International; Suwanee, Ga., USA) with 0.2 μm diameter pores and made a series of dilutions for detection. The precision of this method in terms of recovery rate was evaluated by spiking Ara h 1 standard solutions of 200, 500, and 1000 ng/mL into the food samples. Each concentration was performed three times to ensure the consistency of the response trend, and the recovery rate was calculated as follows:

$$Recovery ( \% ) = \frac{\overline{{C}^{′}} - \overline{{C}_{0}}}{{C}_{S}}$$

where C̅′̅ is the mean Ara h 1 concentration of the spiked samples, C̅₀̅ is the mean Ara h 1 concentration of the blank sample, and C_{S} is the concentration of the standard solution spiked into the sample.

The results in Table 1 show that the spiked recoveries measured by the presented biosensor were consistent with the ELISA kit.

TABLE 1
Determination of Ara h 1 concentration in actual samples
by the exemplary biosensor and an ELISA kit

| Spiked | Measured | Recovery | Measured | Recovery |
|---|---|---|---|---|
| concen- | by bio- | by bio- | by ELISA | by ELISA |
| tration | sensor | sensor | kit | kit |
| (ng/mL) | (ng/mL) | (%) | (ng/mL) | (%) |
| 0 | 116.5 ± 3.5% | | 97.4 ± 3.4% | |
| 200 | 291.5 ± 5.6% | 87.5 | 269.1 ± 4.0% | 85.8 |
| 500 | 566.5 ± 4.9% | 90 | 590.7 ± 5.5% | 98.7 |
| 1000 | 1041.5 ± 4.2% | 92.5 | 1001.3 ± 3.5% | 90.4 |

### Conclusions

A microfluidic biosensor for Ara h 1 detection was developed by using a quencher system with graphene oxide and aptamer-functionalized quantum dots. This fluorescence was “turned off” by a fluorescence resonance energy transfer between the Qdots-aptamer and graphene oxide and “turned on” with the addition of Ara h 1 due to the better association constant between the Qdots-aptamer and Ara h 1 compared to that between the Qdots-aptamer and GO, which led to the release of the Qdots-aptamer from GO, resulting in the recovery of the fluorescence of Qdots. The Qdots-aptamer probes showed superior fluorescent properties. Based on the principle above, an assay for Ara h 1 detection was performed on a microfluidic platform with a miniaturized optical sensor. The results proved that the presented method was capable of the simple, rapid, sensitive, and reliable detection of Ara h 1 with a detection limit of 56 ng/mL. The results also suggested this method had the potential for on-site determination for rapidly detecting food allergens with the flexibility to select aptamers for specifically targeted allergens.

### Example 2

### A Multipurpose PMSA/Paper Microfluidic Biosensor Using Graphene Oxide and Aptamer-Functionalized Quantum Dots for Food Allergens and Toxins Detection

Food safety is a worldwide health concern to both humans and animals and received extensive attention from researchers. Food analysis is requiring rapid, accurate, sensitive and cost-effective methods to monitor and guarantee the safety and quality to fulfill the strict food legislation and consumer demands. Conventional analytical including enzyme-linked immunosorbent assay (ELISA), high-performance liquid chromatography (HPLC), and capillary electrophoresis (CE), are usually time-consuming, laborious and requires skilled technicians, which make these methods not suitable for handheld towards device development to meet the aforementioned market demands. Here, a nano-materials enhanced multipurpose PDMS/paper microfluidic aptasensor was used to accurately detect food allergens and food toxins. Graphene oxide (GO) and specific aptamer-functionalized quantum dots (QDs) were employed as probes, the fluorescence quenching and recovering of the QDs caused by the interaction among GO, aptamer-functionalized QDs and the target protein were investigated to quantitatively analyze the target concentration. The homogenous assay was performed on the PDMS/paper microfluidic chip, which significantly decreased the sample and reagent consumptions and reduced the assay time. Egg white lysozyme, lupine and food toxins, okadaic acid and brevetoxin standard solutions and spiked food samples were successfully assayed by the present aptasensor. Dual-target assay could be completed within 5 min, and superior or comparable sensitivities were achieved when testing the samples with commercial ELISA kits side by side.

The integration of a microfluidics platform in a miniaturized optical analyzer provides a promising way for the rapid, cost-effective, and accurate on-site determination of food allergens and toxins. This biosensor may also be extended to the detection of other food allergens and toxins with a selection of corresponding aptamers. For example, additional allergen- and toxin-binding aptamers are described in Tran et al., 2013; Nadal et al., 2013; Mairal et al., 2014, Amaya-González et al., 2014 and Cruz-Aguado and Penner, 2008, hereby incorporated by reference in their entirety.

Hen egg is known as one of the most common cause of food allergies both in children and adults. Lysozyme of egg origin is one of the main egg white proteins and being increasingly used in the dairy industry as an antibacterial additive to prevent spoilage of many foodstuffs such as cheese and wine, as well as some medicinal products (Benedé et al., 2014). However, lysozyme is a potential food allergen and accounts for 10-20% of egg allergy which may cause immediate or late adverse reactions such as vomiting, nausea, itching, urticarial and so on (Marseglia et al., 2013). Lupine (Lupin) is a legume belongs to a diverse genus of Fabaceae family which is characterized by long flowering spikes (LUPINS.org, 2016). It has been intensively used in food due to its high value in nutrition and can be found in a wide variety of food products including bread, pasta, sauces, beverages and meat based products such as sausages (ASCIA, 2015). It is also increasingly used as a protein replacement for animal proteins such as egg white and milk. However, lupine allergy is on the rise and hidden lupine allergens in food are a critical problem for lupine sensitive individuals since even very low amounts of lupine may trigger allergic reactions, and in severe cases it may lead to life-threatening anaphylaxis (Lupine ELISA Package Insert, 2016). Because of this, lupine allergic persons must strictly avoid the consumption of lupine containing food. Lupine has recently been added to the declaration list of ingredients requiring mandatory indication on the label of foodstuffs within the European Union (Stanojcic-Eminagic, 2010). Harmful algal bloom (HABs) outbreaks have reportedly intensified throughout the world and pose a grave threat to public health and local economies. HAB toxins through food may cause human diseases by releasing several shellfish toxins, including neurotoxic shellfish poison (NSP), diarrheic shellfish poison (DSP), paralytic shellfish poison (PSP), ciguatera fish poison (CFP), etc. (Christian and Luckas, 2008; Lin et al., 2015). NSP typically affects the gastrointestinal and nervous systems and is caused by consumption of contaminated shellfish with brevetoxins primarily produced by the dinoflagellate (Watkins et al., 2008). Okadaic acid (OA) is a marine toxin, which may cause the diarrheic shellfish poisons (DSP) produced by some unicellular algae from plankton and benthic microalgae (Sassolas et al., 2013a). It is difficult to directly identify OA because OA usually does not affect the smell, appearance and the taste of the seafood (Sassolas et al, 2013b).

### Materials and Methods

The design of the aptamers specific to target analytes, namely egg white lysozyme, lupine, okadaic acid and brevetoxin, were selected by referring to Tran et al., 2010; Svobodova et al., 2014; Eissa et al., 2015; and Gu et al., 2016, hereby incorporated by reference in their entirety, and synthesized by IDT technologies (Coralville, Iowa, USA), the sequences of the selected aptamers are listed in Table 2, all of which were modified with biotin at the 5′end.

TABLE 2
Sequences of selected aptamers

| | |
|---|---|
| SEQ ID NO: 2 | 5′-AGC AGC ACA GAG GTC AGA TG GCA GCT |
| ID: Aptamer Sequence | AAG CAG GCG GCT CAC AAA ACC ATT CGC |
| Targeting Lysozyme | ATG CGG C CCT ATG CGT GCT ACC GTG AA- |
| | 3′ |
| SEQ ID NO: 3 | 5′-AGC TGA CAC AGC AGG TTG GTG GGG |
| ID: Aptamer Sequence | GTG GCT TCC AGT TGG GTT GAC AAT ACG |
| Targeting Lupine | TAG GGA CAC GAA GTC CAA CCA CGA GTC |
| | GAG CAA TCT CGA AAT-3′ |
| SEQ ID NO: 4 | 5′-CAG CTC AGA AGC TTG ATC CTA TTT GAC |
| ID: Aptamer Sequence | CAT GTC GAG GGA GAC GCG CAG TCG CTA |
| Targeting Okadaic acid | CCA CCT GAC TCG AAG TCG TGC ATC TG-3′ |
| SEQ ID NO: 5 | 5′-ATA CCA GCT TAT TCA ATT GGC CAC CAA |
| ID: Aptamer Sequence | ACC ACA CCG TCG CAA CCG CGA GAA CCG |
| Targeting Brevetoxin | AAG TAG TGA TCA TGT CCC TGC GTG AGA |
| | TAG TAA GTG CAA TCT-3′ |

Food Lupine ELISA Test Kit and Brevetoxin (NSP) ELISA Kit were purchased from Creative Diagnostics (Shirley, N.Y., USA), Lysozyme ELISA Kit and Okadaic Acid (DSP) ELISA Test Kit were obtained from LifeSpan BioSciences, Inc. (Seattle Wash., USA) and Bioo Scientific Corporation (Austin, Tex., USA), respectively. CdSe Quantum dots modified with covalently attached streptavidin (Qdot® 545 ITK™ Streptavidin Conjugates) were purchased from Invitrogen Life Technologies (Burlington, ON, Canada). Polydimethylsiloxane (PDMS, Sylgard, 184) was obtained from Dow Corning (Midland, Mich., USA), SU-8 photoresist and developer were obtained from MicroChem Corp. (Westborough, Mass., USA). Whatman chromatography paper, graphene oxide, phosphate-buffered saline (PBS), bovine serum albumin (BSA), methanol and all other mentioned chemicals and reagents were purchased from Sigma-Aldrich (Oakville, ON, Canada). Unless otherwise noted, all solutions were prepared with ultrapure Milli-Q water (18.2 MV cm). Eggs, mussels and all other food samples were purchased from grocery stores in Guelph (ON, Canada).

### Preparation of Aptamer-QDs Functionalized GO

The detailed preparation and optimization of aptamer-QDs functionalized GO is described in Example 1. Briefly, biotinylated aptamer of 10 μM in folding buffer (1 mM MgCl2, 1×PBS, pH 7.4) was heated at 85° C. for 5 min. The cooling down aptamer was mixed with streptavidin-conjugated quantum dots of 2 μM to proceed with the covalent linking via streptavidin-biotin interaction. The mixture was then brought to 200 μL with PBS and gently shaken for 12 hours under room temperature (RT) condition. The aptamer-QDs conjugates was then obtained by subjecting to ultrafiltration (Amicon Ultra-0.5 mL centrifugal filters, MWCO 50 kDa, EMD Millipore Inc.) with PBS at 6000 rpm for 15 min, repeated three times. The purified aptamer-QDs conjugates re-suspended in 500 μL of PBS and kept at dark for further use.

### Microfluidic Biochip Fabrication and Signal Capture

The schematic of the PDMS/paper microfluidic chip and the pictures of the real chip are shown in FIG. 6. A high resolution transparency photomask bearing microchannel layout design was firstly drawn by AutoCAD software and printed by Fineline Imaging (Colorado Springs, Colo., USA). A master mold was then prepared using 2025 negative photoresist SU-8 by standard photolithography. A thin layer of SU-8 was spin-coated on the surface of the wafer, followed by prebaking at 65° C. for 3 min and 95° C. for 9 min on a hotplate. Afterwards, the photomask was placed onto the coated silicon wafer and exposed to UV using a UV exposure system (UV-KUB, Kloé, France). A master mold was ready after the post-baking, development and hard-baking. The simple PDMS/paper microfluidic chip consisted of two PDMS layers and a glass slide. The bottom layer of PDMS carried two pairs of wells (φ=3 mm) for housing well-cut chromatography paper (φ=3 mm) with QDs-aptamer-GO coating. The two pairs of wells were designed for dual-target detection with duplicate readouts to reduce the testing error. The top layer of PDMS bearing sample inlet, outlets and associated dispensing channels. Both of the PDMS slabs were created by following the standard soft lithography protocol. Briefly, a mixture of prepolymers of PDMS (10:1 w/w ratio of PDMS and curing agent) was poured onto the master mold at 75° C. for 4 h after degassing. The bottom layer of PDMS slab carrying four reaction wells was punched and bond onto the glass. The wells were filled with 0.1% BSA (w/w) for 10 min, washed with lx PBS and left to dry at RT to reduce the non-specific adsorption of proteins of the PDMS wall (Windvoel et al., 2010). The top layer of PDMS slab was also be punched to form the inlet and outlets. Afterwards, both of these two components underwent the plasma treatment for bonding and the chromatography paper adsorbing specific aptamer bound QDs-GO probes was placed into the wells before bonding. Then a PDMS/paper microfluidic chip was ready for use.

### Preparation of Food Samples

Food sample preparation was conducted by following the procedure indicated in the manual of the commercial kits.

Briefly, fresh egg white was first diluted with sample diluent to make the dilution series (up to 20000-fold) and followed by centrifuging at 4000×g for 10 minutes at 4° C. to remove the particulates. The supernatant was then used for assay.

Mussel tissue was taken off the shells, washed by DI water and excess liquid was drained, followed by homogenization. 0.5 g of homogenized mussel tissue was carefully weighed and added with 2 mL of 50% methanol followed by vortex for 5 min. The mixture was centrifuged at 4000 rpm for 10 min and 0.5 mL of the supernatant was transferred to a new tube, heated at 75° C. for 5 min and followed by centrifugation again for another 10 min at 4000 rpm. Then 50 μL of the final supernatant was ready for use after the addition with 950 μL of 1× Sample Extraction Buffer.

Sausage sample were first well grinded and 1 g of the homogenized sausage was suspended in 20 mL of pre-diluted extraction and sample dilution buffer followed by 15 min of incubation in a water bath at 60° C. with frequent shaking. Afterwards, the sample was centrifuged at 2000×g for 10 min, and the supernatant was ready for assay.

### Assay Procedure

Fluorescence images were taken by a Nikon DS-QiMc microscope camera mounted on the fluorescent microscopy followed by the fluorescent intensity measurement by the Nikon NIS Elements BR version 4.13 software (Nikon Eclipse Ti, Nikon Canada Inc., Mississauga, ON, Canada). All images were taken under the same settings, namely exposure time, magnification,etc.

Food sample detection by commercial kits was conducted by following standard ELISA procedure described in the manual.

### Results and Discussion

### Characterization and Validation

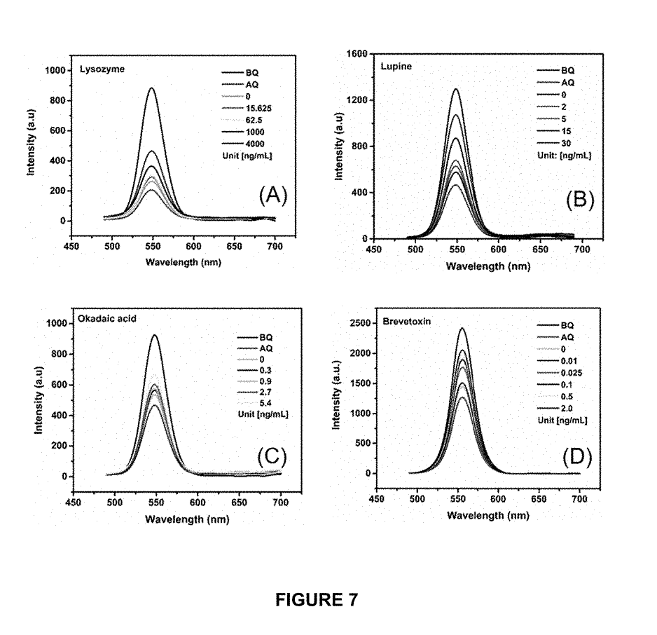

The aptamer-QDs were investigated by dynamic light scattering (DLS) analysis and fluorescence spectra measurement to confirm the conjugations. The detailed procedures can be found in Example 1. The increased mean hydration diameters and zeta ( )potential values of the nanoparticles and a decrease in the fluorescence intensity of the QDs were used to verdict the successful conjugations. Before the on-chip test, the standard solutions of these four analytes were measured on the Cytation 5 Multi-mode Reader (BioTek, Winooski, Vt., USA) to validate the occurrence of sensing events. The results are shown in the FIG. 7, differentiable fluorescence spectra dependent on the sample concentrations were observed.

### On-Chip Test

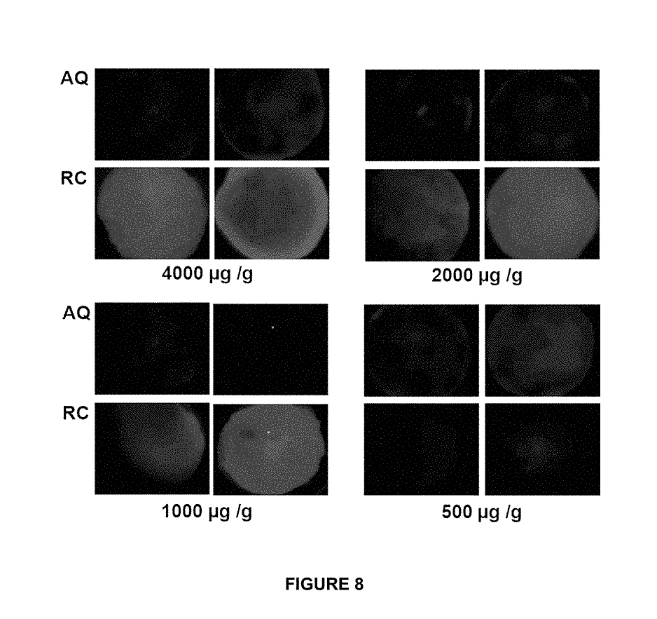

10 μL of standard solutions or samples were loaded into the central well of the microfluidic chip and dispensed into the four reaction wells by capillary force and contact the GO-aptamer-QDs coated chromatography paper. The fluorescence intensities after quenching and recovery were scanned and recorded by the fluorescence microscope, the intensity differences in between were employed to determine the concentrations of the target. Pieces of chromatography paper with the same aptamer-specific GO-QDs were placed in two wells for duplication. Hence the designed PDMS/paper microfluidic chip was able to achieve the dual-target detection with duplication. FIG. 8 gives an example of the fluorescence images taken after quenching (AQ) and after recovery (RC) by assaying egg white lysozyme of various concentrations. The mean fluorescence intensity of the overall of the reaction well was then analyzed via the Nikon NIS Elements BR software.

The standard curves were obtained by plotting the mean fluorescence intensity for each stand on the Y-axis against the target concentrations on the X-axis, a linear fit curve were created through the points. The fit curves were presented in the plots shown in FIG. 9, the linear regressions of 0.9469, 0.9839, 0.975 and 0.9838 were calculated and obtained for egg white lysozyme, lupine and food toxins, okadaic acid and brevetoxin standard solutions, respectively. The calculated limits of detection (Thomsen et al., 2003) based on the standard curves are 343 ng/mL, 2.5 ng/mL, 0.4 ng/mL and 0.56 ng/mL, respectively. These limits of detection by the present aptasensor are superior or comparable to those claimed by the ELISA kits (16 ng/mL, 30 ng/mL, 200 ng/mL and 0.16 ng/mL).

### Food Samples Detection

Spiked food samples were detected by both the on-chip method and the ELISA kits for egg white lysozyme, lupine, okadaic acid and brevetoxin to investigate the accuracy of the on-chip method. Standard solutions were firstly assayed to obtain the standard curves, as shown in FIG. 9. The precision of this method in terms of recovery rate was evaluated by detecting spiked food samples, fresh egg white, mussels, sausages and breads. Each concentration was performed three times to ensure the consistency of the response trend and the recovery rate was calculated as follows:

$$Recovery = \frac{\overline{{C}^{′}} \cdot {V}^{′} - \overline{{C}_{0}} \cdot {V}_{0}}{{C}_{S} \cdot {V}_{S}} \times 100 \%$$

where C̅′̅ and C̅₀̅ are the mean target concentration of the spiked sample and the blank sample, respectively. C_{S} is the concentration of the standard solution spiked into the sample. V′, V₀, and V_{S} are the volumes of the final spiked sample, blank sample and the standard spiking solution, respectively.

Samples of lysozyme, lupine, okadaic acid and brevetoxin ranging from 0˜4000 ppm, 0˜30 ppm, 0˜16.2 ppm and 0˜2 ppm, respectively, were spiked and tested. The results in Table 3 show that the spiked recoveries measured by the present aptasensor were consistent with ELISA kits. As listed in the table, recovery rates of (91.8±2.73) %˜(110.18±3.54) %, (89.25±8.30) %˜(116.68±10.52) %, (89.63±7.33) %˜(105.00±11.46) % and (88.00±9.17) %˜(112.53±12.22) % were measured in egg white lysozyme, lupine, okadaic acid and brevetoxin for the spiked food samples.

TABLE 3
Determination of target analytes concentration in
spiked food samples by aptasensor and ELISA kits

| | Spiked | Recovery | Recovery |
|---|---|---|---|
| | concen- | by aptasen- | by ELISA |
| | tration | sor (%) | (%) |
| Analyte | (ppm or μg/g) | (mean ± S.D.) | (mean ± S.D.) |
| Lysozyme | 0 | — | — |
| | 500 | 91.80 ± 2.73 | 96.34 ± 4.26 |
| | 1000 | 93.57 ± 17.08 | 97.61 ± 6.23 |
| | 2000 | 110.18 ± 3.54 | 104.56 ± 1.71 |
| | 4000 | 107.25 ± 7.94 | 105.53 ± 6.51 |
| Lupine | 0 | — | — |
| | 2 | 89.25 ± 8.30 | 96.60 ± 6.55 |
| | 5 | 90.4 ± 4.23 | 92.54 ± 7.26 |
| | 15 | 101.73 ± 7.14 | 107.05 ± 1.67 |
| | 30 | 116.68 ± 10.52 | 109.02 ± 3.43 |
| Okadaic | 0 | — | — |
| acid | 0.2 | 105.00 ± 11.46 | 110.00 ± 8.66 |
| | 0.8 | 96.25 ± 8.59 | 92.5. ± 5.73 |
| | 8.1 | 89.63 ± 7.33 | 93.58 ± 6.38 |
| | 16.2 | 104.29 ± 4.80 | 106.85 ± 3.58 |
| Brevetoxin | 0 | — | — |
| | 0.05 | 106.67 ± 8.33 | 105.88 ± 6.00 |
| | 0.1 | 88.00 ± 9.17 | 94.96 ± 7.90 |
| | 0.25 | 112.53 ± 12.22 | 113.18 ± 4.86 |
| | 2 | 108.07 ± 3.05 | 103.45 ± 4.50 |

### Conclusions

A multipurpose PDMS/paper microfluidic aptasensor was developed for the analysis of target analytes. This example demonstrates the detection and quantification of food allergens (egg white lysozyme, lupine) and seafood toxins (okadaic acid and brevetoxin) using the aptasensor.

The PDMS/paper microfluidic aptasensor utilized graphene oxide as quencher which can quench the fluorescence of quantum dots conjugated onto the target-specific aptamers. The fluorescence is recovered in the presence of target and its intensity is proportional to the concentration of the target. A significantly decreased sample volume (10 μL) was needed and dual-target detection with duplicated results could be achieved in a single test within 5 min to reduce the chance of error. Limit of detection of this sensing platform are 343 ng/mL, 2.5 ng/mL, 0.4 ng/mL and 0.56 ng/mL with the linear regressions of 0.9469, 0.9839, 0.975 and 0.9838 for egg white lysozyme, lupine and food toxins, okadaic acid and brevetoxin standard solutions, respectively.

The experimental results by this aptasensor demonstrated remarkable sensitivity and selectivity. Compared to ELISA, which is typically used to detect food allergens and toxins in a centralized lab, this biosensor and associated method is rapid, highly sensitive, selective, less expensive, environmentally friendly, and easy to handle. The present method provides a promising way for the rapid, cost-effective, and accurate determination of food allergens or seafood toxins and also presented its potential of on-site determination capability as well as the flexibility for specifically targeted allergens and toxins by selecting corresponding aptamer. An image intensity analyzer may also be embedded in this microfluidic aptasensor to build a handheld detection device.

All publications, patents and patent applications are herein incorporated by reference in their entirety to the same extent as if each individual publication, patent or patent application was specifically and individually indicated to be incorporated by reference in its entirety.

### REFERENCES

Abachi, F. and Noureini, S. K., 2011. Clin. Biochem. 44, S257.
Algar, W. R., Tavares, A. J., Krull, U. J., 2010. Anal. Chim. Acta. 673(1), 1-25.
Alves, R. C., Barroso, M. F., González-Garcia, M. B., Oliveira, M. B. P. P., Delerue-Matos, M. C., 2015. New trends in food allergens detection: towards biosensing strategies. Crit. Rev. Food Sci. Nutr. DOI: 10.1080/10408398.2013.831026.
Al-Muhsen, S., Clarke, A. E., Kagan, R. S., 2003. CMAJ 168(10), 1279-1285.
Amaya-González, S., de-los-Santos-Álvarez, N., Miranda-Ordieres, A. J., Lobo-Castañón, M. J., 2013. Sensors 13(12), 16292-16311.
Amaya-González, S., de-los-Santos-Álvarez, N., Miranda-Ordieres, A. J., Lobo-Castañín, M.J., 2014. Anal. Chem. 86(5), 2733-2739.
ASCIA, 2015. http://www.allergy.org.au/health-professionals/papers/lupin-food-allergy.
Asensio, L., González, I., Garcia, T., Martin, R., 2008. Food Control 19(1), 1-8.
Benedé, S., Perez-Rangel, I., Lozano-Ojalvo, D., Molina, E., Ibanez, M. D., López-Fandiño, R., López-Expósito, I., 2014. Arch. Allergy Immunol. 165(2), 83-90.
Burks, A. W., Williams, L. W., Helm, R. M., Connaughton, C., Cockrell, G., O'Brien, T. 1991. J. Allergy Clin. Immunol. 88(2), 172-179.
Bogomolova, A., Aldissi, M., 2011. Biosens. Bioelectron. 26(10), 4099-4103.
Christian, B., Luckas, B., 2008. Anal. Bioanal. Chem. 391, 117-134.
Cruz-Aguado, J. A. and Penner, G., 2008. J. Agric. Food Chem. 56(22), 10456-10461.
Cui, Z. Q., Ren, Q., Wei, H. P., Chen, Z., Deng, J. Y., Zhang, Z. P., Zhang, X. E., 2011. Nanoscale 3(6), 2454-2457.

Di Stefano, V., Avellone, G., Bongiorno, D., Cunsolo, V., Muccilli, V., Sforza, S., Dossena, A., Drahos, L., Vékey, K., 2012. J. Chromatogr. A. 1259, 74-85.
Eissa, S., Siaj, M., Zourob, M., 2015. Biosens. Bioelectron. 69, 148-154.
Frasco, M. F., Chaniotakis, N., 2010. Anal. Bioanal. Chem. 396(1), 229-240.
Geim, A. K., Novoselov, K. S., 2007. Nat. Mater. 6(3), 183-191.
Gu, H., Duan, N., Wu, S., Hao, L., Xia, Y., Ma, X., Wang, Z., 2016. Sci. Rep. 6, 21665.
Gutteridge A, Thornton J. M., 2005. Trends Biochem. Sci. 30, 622-629.
He, S. J., Song, B., Li, D., Zhu, C. F., Qi, W. P., Wen, Y.Q., Wang, L. H., Song, S. P., Fang, H. P., Fan, C. H., 2010. Adv. Funct. Mater. 20, 453-459.
He, Y., Lu, M., Cao, J., Lu, H. P., 2012. ACS Nano 6, 1221-1229
Huang, P. J. J., Liu, J., 2012. Small 8(7), 977-983.
Koppelman, S. J., Wensing, M., Ertmann, M., Knulst, A. C., Knol, E. F., 2004. Clin. Exp. Allergy 34, 583-590.
Levy, M., Cater, S. F., Ellington, A. D., 2005. Chem. Bio. Chem. 6(12), 2163-2166.
Li, S., Aphale, A. N., Macwan, I. G., Patra, P. K., Gonzalez, W. G., Miksovska, J., Leblanc, R. M., 2012. ACS Appl. Mater. Interfaces 4(12), 7069-7075.
Lin, C., Liu, Z. S., Tan, C. Y., Guo, Y. P., Li, L., Ren, H. L., Li, Y. S., Hu, P., Gong, S., Zhou, Y., Lu, S. Y., 2015. Environ. Sci. Pollut. Res. 22(2), 1545-1553.
Lu, C. H., Yang, H. H., Zhu, C. L., Chen, X., Chen, G. N., 2009. Angew. Chem. 121(26), 4879-4881.
Lu, Z., Chen, X., Wang, Y., Zheng, X., Li, C. M., 2015. Microchim. Acta 182(3-4), 571-578.
LUPINS.org, 2016. Information resource portal for lupins. http://www.lupins.org/lupins/Lupine
ELISA Package Insert, 2016. Creative Diagnostics, Shirley, N.Y., USA. http://img.creative-diagnostics.com/pdf/DEIA294%20Lupin%20ELISA%20Kit.pdf
Mairal, T., Nadal, P., Svobodova, M., O'Sullivan, C. K., 2014. Biosens. Bioelectron. 54, 207-210.
Marseglia, A. L., Castellazzi, A. M., Valsecchi, C. H., Licari, A. M., Piva, G. I., Rossi, F., Fiorentini, L., Marseglia, G. L, 2013. Eur. J. Nutr. 52(3), 877-883.
Mohammed, M. I., Desmulliez, M. P. Y., 2013. Microsyst. Technol., 19(6), 809-818.
Monaci, L., De Angelis, E., Bavaro, S. L., Pilolli, R., 2015. Food Addit. Contam. Part A 32(10), 1607-1616.
Morales-Narváez, E., Pérez-López, B., Pires, L. B., Merkoçi, A., 2012. Carbon 50(8), 2987-2993.
Nadal, P., Svobodova, M., Mairal, T., O'Sullivan, C. K., 2013. Anal. Bioanal. Chem. 405(29), 9343-9349.
Nollet, L. M., Toldrá, F., 2012. 3rd eds., Food analysis by HPLC, Vol. 100; CRC Press Book.
Peeters, M., Jiménez-Monroy, K. L, Libert, C., Eurlings, Y., Cuypers, W., Wackers, G., Duchateau, S., Robaeys, P., Nesládek, M., van Grinsven, B., Pérez-Ruiz, E., Lammertyn, J., Losada-Pérez, P., Wagner, P., 2014. J. Biosens. Bioelectron. 5, 155.
Peng, J., Song, S., Xu, L., Ma, W., Liu, L., Kuang, H., Xu, C., 2013. Int. J. Environ. Res. Public Health 10(7), 2897-2905.
Pomés, A., Helm, R. M., Bannon, G. A., Burks, A. W., Tsay, A., Chapman, M.D., 2003. J. Allergy Clin. Immunol. 111(3), 640-645.
Rodríguez-Lázaro, D., Cook, N., Hernández, M., 2013. Curr. Issues Mol. Biol. 15(2), 39-44.
Sassolas, A., Hayat, A., Catanante, G., Marty, J.L., 2013. Talanta 105, 306-316. [Sassolas et al., 2013a]
Sassolas, A., Catanante, G., Hayat, A., Stewart, L. D., Elliott, C. T., Marty, J. L., 2013. Food Control 30(1), 144-149. [Sassolas et al., 2013b]
Song, E., Cheng, D., Song, Y., Jiang, M., Yu, J., Wang, Y., 2013. Biosens. Bioelectron. 47, 445-450.
Stanojcic-Eminagic, S., 2010. Food and agricultural import regulations and standards-Narrative FAIRS Country Report: Bosnia and Herzegovina. GAIN (Global Agriculture and Information Network) report Number: BK1005. United States Department of Agriculture (USDA) Foreign Agricultural Service. Washington D.C., USA; Food Standards Agency.
Svobodova, M., Mairal, T., Nadal, P., Bermudo, M. C., O'Sullivan, C. K., 2014. Food Chem. 165, 419-423.
Swathi, R. S., Sebastian, K. L., 2009. J. Chem. Phys. 130(8), 086101.
Thomsen, V., Schatzlein, D., Mercuro, D., 2003. Spectroscopy 18 (12), 112-114.
Tran, D. T., Knez, K., Janssen, K. P., Pollet, J., Spasic, D., Lammertyn, J., 2013. Biosens. Bioelectron. 43, 245-251.
Tran, D. T., Janssen, K. P., Pollet, J., Lammertyn, E., Anne, J., Van Schepdael, A., Lammertyn, J., 2010. Molecules 15(3), 1127-1140.
van Hengel, A. J., 2007. Anal.Bioanal. Chem. 389(1), 111-118.
Wang, Y., Deng, R., Zhang, G., Li, Q., Yang, J., Sun, Y., Li, Z., Hu, X., 2015. J. Agric. Food Chem. 63(8), 2172-2178.
Wang, Y., Li, Z., Hu, D., Lin, C. T., Li, J., Lin, Y., 2010. J. Am. Chem. Soc. 132(27), 9274-9276.
Watkins, S. M., Reich, A., Fleming, L. E., Hammond, R., 2008. Mar. Drugs 6(3), 431-455.
Weng, X., Zhao, W., Neethirajan, S., Duffield, T., 2015. J. Nanobiotechnol. 13(1), 13.
Windvoel, T., Mbanjwa, M., Mokone, N., Mogale, A., Land, K., 2010. World Acad. Sci. Eng. Technol. 61, 354-356.
Zeng, Q., Zhang, Y., Liu, X., Tu, L., Kong, X., Zhang, H., 2012. Chem. Commun. 48, 1781-1783.
Zhang, M., Yin, B. C., Tan, W., Ye, B. C., 2011. Biosens. Bioelectron. 26(7), 3260-3265.
Zhang, W. J., Cai, Q., Guan, X., Chen, Q., 2015. Food Chem. 174, 547-552.
Zimmermann, M., Schmid, H., Hunziker, P., Delamarche, E., 2007. Lab Chip 7(1), 119-125.
Zuo, P., Li, X., Dominguez, D. C., Ye, B. C., 2013. Lab Chip 13(19), 3921-3928.

1. A probe composition comprising:
a probe comprising an allergen- or toxin-binding molecule conjugated to a fluorophore, and
graphene oxide,
wherein the probe adheres to graphene oxide such that the fluorophore is quenched through fluorescence energy resonance transfer (FRET) and the probe dissociates from graphene oxide when bound to a target allergen or toxin.

2. The probe composition of claim 1, wherein the allergen- or toxin-binding molecule is an aptamer or antibody and the fluorophore is a quantum dot.

4. The probe composition of claim 1, wherein the allergen-binding molecule selectively binds to an allergen selected from peanut allergens, egg allergens, legume allergens, milk allergens, seafood allergens, mustard allergens, sesame allergens, soy allergens, tree nut allergens and wheat allergens.

5. The probe composition of claim 4, wherein the peanut allergen is Ara h 1, the egg allergen is lysozyme, and/or the legume allergen is lupine.

6. The probe composition of claim 5, wherein the allergen-binding molecule is an aptamer comprising a nucleic acid molecule with a nucleic acid sequence having at least 90% sequence identity to SEQ ID NO: 1, SEQ ID NO: 2 or SEQ ID NO: 3.

7. The probe composition of claim 1, wherein the toxin-binding molecule selectively binds to a seafood toxin selected from okadaic acid and brevetoxin.

8. The probe composition of claim 7, wherein the toxin-binding molecule is an aptamer comprising a nucleic acid molecule with a nucleic acid sequence having at least 90% sequence identity to SEQ ID NO: 4 or 5.

9. A biosensor comprising one or more of the probe compositions of claim 1 and a microfluidic device.

10. The biosensor of claim 9, wherein the microfluidic device comprises a first inlet for receiving the probe composition and a second inlet for receiving a test sample, wherein the first inlet and second inlet are in fluid communication with a mixing channel and the mixing channel is in fluid communication with a sensing well.

11. The biosensor of claim 9, wherein the microfluidic device comprises a reaction well containing the probe composition and a sample well for receiving a test sample, wherein the sample well is in fluid communication with a sample dispensing channel extending from the sample well to the reaction well.

12. The biosensor of claim 11, wherein the probe composition is in contact with a substrate in the reaction well.

13. The biosensor of claim 11, comprising a plurality of sample dispensing channels extending radially from the sample well to a plurality of reaction wells.

14. The biosensor of claim 13, wherein a first reaction well comprises a first probe comprising a first allergen- or toxin-binding molecule and a second reaction well comprises a second probe comprising a second allergen- or toxin-binding molecule, wherein the first probe and second probe bind to different allergens or toxins or to the same allergens or toxins.

15. The biosensor of claim 11, further comprising an optical detector, wherein the optical detector comprises an excitation light source and a photodiode for measuring fluorescence of the fluorophore.

16. A method for detecting and/or quantifying a concentration of a target allergen or toxin in a sample, the method comprising:
contacting the sample with the probe composition of claim 1; and
detecting a level of fluorescence of the probe composition in contact with the sample, wherein the level of fluorescence is proportional to the concentration of target allergen or toxin in the sample.

17. The method of claim 16, further comprising detecting a level of fluorescence of the probe composition prior to contacting the probe composition with the sample and detecting a change in a level of fluorescence of the probe composition after contacting the probe composition with the sample.

18. The method of claim 16, further comprising comparing the level of fluorescence of the probe composition in contact with the sample to one or more control levels, wherein each control level is indicative of a pre-determined concentration of the target allergen or toxin in a control sample.

19. The method of claim 16, comprising contacting the sample with the probe composition on a microfluidic device.

20. The method of claim 16, wherein the method comprises contacting the sample with a plurality of probe compositions and detecting and/or quantifying the concentration of a plurality of target allergens and/or toxins in the sample.


SEQUENCE LISTING (5 sequences)

SEQ ID NO: 1 - DNA, 77 bp, Artificial Sequence
  Synthetic construct
tcgcacattccgcttctaccgggggggtcgagcgagtgagcgaatctgtgggtgggccgtaagtccgtgtgtgcgaa

SEQ ID NO: 2 - DNA, 80 bp, Artificial Sequence
  Synthetic construct
agcagcacagaggtcagatggcagctaagcaggcggctcacaaaaccattcgcatgcggccctatgcgtgctaccgtgaa

SEQ ID NO: 3 - DNA, 93 bp, Artificial Sequence
  Synthetic construct
agctgacacagcaggttggtgggggtggcttccagttgggttgacaatacgtagggacacgaagtccaaccacgagtcgagcaatctcgaaat

SEQ ID NO: 4 - DNA, 80 bp, Artificial Sequence
  Synthetic construct
cagctcagaagcttgatcctatttgaccatgtcgagggagacgcgcagtcgctaccacctgactcgaagtcgtgcatctg

SEQ ID NO: 5 - DNA, 96 bp, Artificial Sequence
  Synthetic construct
ataccagcttattcaattggccaccaaaccacaccgtcgcaaccgcgagaaccgaagtagtgatcatgtccctgcgtgagatagtaagtgcaatct